(19) United States
(12) Patent Application Publication
(10) Pub. No.: US 2010/0261695 A1
(43) Pub. Date: Oct. 14, 2010

(54) TREATMENT OF ONYCHOMYCOSIS AND RELATED COMPOSITIONS

(76) Inventor: Reza BABAPOUR, Beverly Hills, CA (US)

Correspondence Address: KAREN S. CANADY;CANADY & LORTZ LLP, 4201 Wilshire BLV Suite 622, LOS ANGELES, CA, 90010, US

(21) Appl. No.: 12/757,881

(22) Filed: Apr. 9, 2010

Related U.S. Application Data
(60) Provisional application No. 61/212,320, filed on Apr. 9, 2009.

(51) Int. Cl. A61K 31/555; A61K 31/4418; A61K 31/496; A61K 31/506; A61K 31/5375; A61P 31/10

(52) U.S. Cl. 514/188; 514/345; 514/254.07; 514/275; 514/239.5

(57) ABSTRACT

The present invention generally relates to the treatment of onychomycosis. More specifically, it relates to combination therapies for the treatment of onychomycosis and related compositions. In a composition aspect, the present invention provides a composition for the treatment of onychomycosis, wherein the composition includes Ciclopirox and at least one other antifungal agent. In a method aspect, the present invention provides a method for treating onychomycosis in a patient suffering from the disease. The method involves topically administering a composition to at least one toe or fingernail of the patient and the composition includes Ciclopirox and at least one other antifungal agent.

This application claims the benefit of U.S. provisional patent application No. 61/212,320, filed Apr. 9, 2009, the entire contents of which are incorporated herein by reference.

### FIELD OF THE INVENTION

The present invention generally relates to the treatment of onychomycosis. More specifically, it relates to combination therapies for the treatment of onychomycosis and related compositions.

### BACKGROUND OF THE INVENTION

Ciclopirox (PENLAC™, Dermik) 8% solution has been approved for the treatment of onychomycosis in the United States and Canada; it is the only topical antifungal approved for such treatment in Canada. While Ciclopirox shows a degree of efficacy against onychomycosis for certain patient populations, it has proven not to be universally effective against the illness.

Accordingly, there is still a need for the development of new onychomycosis treatments and compositions that are related to those treatments.

### SUMMARY OF THE INVENTION

The present invention generally relates to the treatment of onychomycosis. More specifically, it relates to combination therapies for the treatment of onychomycosis and related compositions.

In a composition aspect, the present invention provides a composition for the treatment of onychomycosis, wherein the composition includes Ciclopirox and at least one other antifungal agent.

In a method aspect, the present invention provides a method for treating onychomycosis in a patient suffering from the disease. The method involves topically administering a composition to at least one toe or fingernail of the patient, and the composition includes Ciclopirox and at least one other antifungal agent.

### DETAILED DESCRIPTION OF THE INVENTION

### Definitions

“Ciclopirox” refers to 6-cyclohexyl-1-hydroxy-4-methyl-2(1H)-pyridone.

“Ciclopirox olamine” refers to the 2-aminoethanol salt of Ciclopirox.

The present invention relates to combination therapies for the treatment of onychomycosis and related compositions. The compositions include Ciclopirox and at least a second antifungal agent. In certain cases, the compositions may include Ciclopirox, a second antifungal agent and a third antifungal agent. The combination therapies involve the topical application of compositions according to the present invention and optionally include the oral administration of an antifungal agent. The optional oral agent may be the same as included in the Ciclopirox containing composition or different.

The second or third, etc. antifungal agent included in the composition is typically selected from the following list of agents: terbinafine; itraconazole; ketoconazole; fluconazole; derivatives of fluconazole; oxiconazole; sulconazole; clotrimazole; miconazole; econazole; azanidazole; bifonazole; butoconazole; chlormidazole; fenticonazole; imazalil; isoconazole; neticonazole; sertaconazole; tioconazole; naftifine; griseofulvin; amorolfine; sodium pyrithione, bifonazole/urea; and, propylene glycol-urea-lactic acid.

Where either bifonazole/urea or propylene glycol-urea-lactic acid are included in the composition with Ciclopirox, the concentration of urea in the composition is typically greater than 10%. In other cases, the concentration of urea is between 10% and 60%, 20% and 60%, 25% and 55%, or 35% and 50% inclusive.

The composition may further optionally include a penetration enhancer such as acetyl cysteine. It may further optionally include a penetration synergist such as urea. (For a discussion of penetration enhancers and penetration synergists, see U.S. Pat. No. 6,042,845, which is incorporated-by-reference into this document for all purposes.)

The composition is typically a solution, cream, ointment, foam or spray which is 8% in Ciclopirox, although any concentration that produces a desirable pharmaceutical effect is suitable. Nonlimiting examples of other Ciclopirox concentrations include a 5%, 6%, 7%, 9%, 10%, and 11% solution or cream.

Where a single additional antifungal agent is included in the composition (e.g., itraconazole), it is typically included at a concentration of 0.5%, 1%, 1.5%, 2%, 2.5%, 3%, 3.5%, 4%, 4.5% or 5% in the solution or cream.

Where two additional antifungal agents are included in the composition (e.g., itraconazole and terbinafine), their combined concentration is typically 0.5%, 1%, 1,5%, 2%, 2.5%, 3%, 3.5%, 4%, 4.5% or 5% in the solution or cream.

A nonlimiting list of other agents that may be included in the composition is as follows: cetyl alcohol; cocamide DEA; lactic acid; mineral oil; myristyl alcohol; octyldodecanol; polysorbate 60; purified water; sorbitan monostearate; stearyl alcohol; and benzyl alcohol (1%) as a preservative.

The treatment of onychomycosis involves the topical application of a composition of the present invention to a toe or fingernail of a human or other animal presenting symptoms of the disease.

The treatment may further include the oral administration of an antifungal in conjunction with the topical administration of the composition to a target toe or fingernail. A nonlimiting example of such a regimen is the topical application of a composition including Ciclopirox and itraconazole in conjunction with the oral administration of itraconazole.

Where the oral administration of an antifungal is used, the antifungal is provided at a significantly lower dose than typically administered for the treatment of onychomycosis—e.g., 90%, 80%, 70%, 60%, 50%, 40%, 30%, 20% or 10% of the typically administered dose.

One objective for the treatment involving the oral administration of a reduced amount of an antifungal agent is the significant reduction of patent side effects, which typically accompany the oral administration of certain antifungal agents—e.g., nausea, abdominal pain and rash.

For instance, using standard metrics for determining the severity of nausea, abdominal pain or rash, one will typically see at least a 10% reduction in side effect severity utilizing the combination therapy employing conjunctive oral administration of an antifungal agent according to the present invention vis-à-vis the simple oral administration of the antifungal agent at a therapeutically effective dose. Oftentimes, one will see at least a 20% reduction, 30% reduction, 40% reduction or 50% reduction in side effect severity.

From the foregoing it will be appreciated that, although specific embodiments of the invention have been described herein for purposes of illustration, various modifications may be made without deviating from the spirit and scope of the invention. Accordingly, the invention is not limited except as by the appended claims.

1. A composition for the treatment of onychomycosis, wherein the composition comprises: Ciclopirox and at least one other antifungal agent.

2. The composition according to claim 1, wherein the at least one other antifungal agent is selected from the group consisting of: terbinafine; itraconazole; ketoconazole; fluconazole; derivatives of fluconazole; oxiconazole; sulconazole; clotrimazole; miconazole; econazole; azanidazole; bifonazole; butoconazole; chlormidazole; fenticonazole; imazalil; isoconazole; neticonazole; sertaconazole; tioconazole; naftifine; griseofulvin; amorolfine; sodium pyrithione, bifonazole/urea; and, propylene glycol-urea-lactic acid.

3. The composition according to claim 2, wherein the at least one other antifungal agent is selected from the group consisting of terbinafine, itraconazole and fluconazole.

4. The composition according to claim 3, wherein the at least one other antifungal agent is itraconazole.

5. The composition according to claim 4, wherein only one antifungal agent other than Ciclopirox is including in the composition.

6. A method for treating onychomycosis in a patient suffering from the disease, wherein the method comprises topically administering a composition to at least one toe or fingernail of the patient, wherein the composition comprises Ciclopirox and at least one other antifungal agent.

7. The method according to claim 6, wherein the at least one other antifungal agent is selected from the group consisting of terbinafine; itraconazole; ketoconazole; fluconazole; derivatives of fluconazole; oxiconazole; sulconazole; clotrimazole; miconazole; econazole; azanidazole; bifonazole; butoconazole; chlormidazole; fenticonazole; imazalil; isoconazole; neticonazole; sertaconazole; tioconazole; naftifine; griseofulvin; amorolfine; sodium pyrithione, bifonazole/urea; and, propylene glycol-urea-lactic acid.

8. The method according to claim 7, wherein only one antifungal agent other than Ciclopirox is including in the composition, and wherein the agent is itraconazole.
